(19) United States
(12) Patent Application Publication
(10) Pub. No.: US 2024/0407730 A1
(43) Pub. Date: Dec. 12, 2024

(54) ELECTROANATOMICAL MAPPING ENABLE DILATOR AND SHEATH WITH ELECTRICAL COUPLING

(71) Applicant: Boston Scientific Medical Device Limited, Galway (IE)

(72) Inventors: Lauren Koon, Mississauga (CA); Eduardo Moriyama, Richmond (CA); Gareth Davies, Toronto (CA); Matthew L. Choi, Markham (CA); Charlene Leung, Scarborough (CA); Jackie Leung, Richmond Hill (CA)

(21) Appl. No.: 18/734,697

(22) Filed: Jun. 5, 2024

Related U.S. Application Data
(60) Provisional application No. 63/507,373, filed on Jun. 9, 2023.

(51) Int. Cl. A61B 5/00; A61B 5/06; A61B 5/287; A61B 5/367; A61B 17/34; A61M 25/00

(52) U.S. Cl. CPC A61B 5/6852; A61B 5/066; A61B 5/287; A61B 5/367; A61B 17/3496; A61M 25/0097

(57) ABSTRACT

A medical system includes a sheath having an elongate body. The elongate body includes a proximal portion and a distal portion. One or more sheath electrodes is located on the distal portion. A handle is attached to the proximal portion. The handle includes one or more first couplers capable of electrically coupling to a control system. The system includes a dilator including an elongate body having a proximal portion and a distal portion. One or more dilator electrodes are located on the distal portion. A hub is attached to the proximal portion of the dilator and includes one or more second couplers electrically coupled to the one or more dilator electrodes. The one or more first couplers is configured to electrically couple with the one or more second couplers.

### CROSS-REFERENCE TO RELATED APPLICATIONS

The present application claims priority to U.S. Provisional Patent Application No. 63/507,373 entitled “ELECTROANATOMICAL MAPPING ENABLED DILATOR AND SHEATH WITH ELECTRICAL COUPLING,” filed Jun. 9, 2023, the entirety of which is incorporated herewith.

### TECHNICAL FIELD

The present invention relates generally to methods and devices usable within the body of a patient. More specifically, the present invention is concerned with an apparatus pertaining to an electroanatomical mapping enabled dilator and sheath having an integrated electrical coupling.

### BACKGROUND

Electroanatomical mapping (EAM) is an increasingly prevalent technology useful during in vivo procedures. It enables physicians to identify anatomical regions of the heart and patterns of electrical activation. This is especially useful when treating arrhythmias. Devices that are compatible with EAM systems allow operators to localize them and more easily target specific regions for treatment, allowing for better workflow, better treatment efficacy, and shorter procedure time.

Typically, each EAM device requires a cable to connect to the EAM system. This can result in cumbersome manipulation of the device and a challenging workspace to manage with several cables for the procedure's devices.

### SUMMARY

Example 1 is a medical system including a sheath having an elongate body. The elongate body includes a proximal portion and a distal portion. One or more sheath electrodes is located on the distal portion. A handle is attached to the proximal portion. The handle includes one or more first couplers capable of electrically coupling to a control system. The system includes a dilator including an elongate body having a proximal portion and a distal portion. One or more dilator electrodes are located on the distal portion. A hub is attached to the proximal portion of the dilator and includes one or more second couplers electrically coupled to the one or more dilator electrodes. The one or more first couplers is configured to electrically couple with the one or more second couplers.

Example 2 is the system of Example 1 wherein the sheath elongate body includes a lumen extending from the proximal portion to the distal portion and is configured to receive the dilator.

Example 3 is the system of any of Examples 1 or 2 wherein the hub includes an upper surface, and the one or more second couplers extends from the upper surface.

Example 4 is the system of any of Examples 1-3 wherein the one or more first couplers and the one or more second couplers have corresponding shapes.

Example 5 is the system of any of Examples 1-3 wherein a cross-section of the one or more first couplers and the one or more second couplers is symmetrical.

Example 6 is the system of any of Examples 1-3 wherein a cross-section of the one or more first couplers and the one or more second couplers is asymmetrical.

Example 7 is the system of Example 6 wherein the cross-section includes one or more concave portion and one or more convex portion.

Example 8 is the system of any of Examples 1-7 wherein electrical connection is made between the one or more dilator electrodes and the control system when the one or more first couplers couples with the one or more second couplers.

Example 9 is the system of any of Examples 1-8 wherein the one or more first couplers or the one or more second couplers includes a conductive surface.

Example 10 is the system of any of Examples 1-9 wherein the one or more first couplers or the one or more second couplers includes an insulated housing configured to confine a brush.

Example 11 is the system of any of Examples 1-9 wherein the one or more first couplers or the one or more second couplers includes a contact that is proud of an insulated surface.

Example 12 is the system of any of Examples 1-11 wherein the one or more first couplers or the one or more second couplers includes a plurality of insulated regions.

Example 13 is the system of any of Examples 1-12 wherein the control system is configured to determine position of the dilator with respect to the sheath.

Example 14 is the system of any of Examples 1-13 further comprising a display for displaying one or more anatomical images, parameters, and positioning information.

Example 15 is the system of any of Examples 1-14 wherein the one or more sheath electrodes includes four sheath electrodes and the one or more dilator electrodes includes two electrodes.

Example 16 is a medical system having a sheath including an elongate body. The sheath elongate body includes a proximal portion and a distal portion. One or more sheath electrodes are located on the distal portion. A handle is attached to the proximal portion. The sheath includes one or more first couplers capable of electrically coupling to a control system. The system includes a dilator having an elongate body. The dilator elongate body includes a proximal portion and a tapered distal portion. One or more dilator electrodes are located on the tapered distal portion. A hub is attached to the proximal portion of the dilator. One or more second couplers are located on the hub. The one or more first couplers are configured to electrically couple with the one or more second couplers.

Example 17 is the system of Example 16 wherein the sheath elongate body includes a lumen extending from the proximal portion to the distal portion and is configured to receive the dilator.

Example 18 is the system of Example 16 wherein the hub includes an upper surface, and the one or more second couplers extends from the upper surface.

Example 19 is the system of Example 16 wherein the one or more first couplers and the one or more second couplers have corresponding shapes.

Example 20 is the system of Example 16 wherein a cross-section of the one or more first couplers and the one or more second couplers is symmetrical.

Example 21 is the system of Example 16 wherein a cross-section of the one or more first couplers and the one or more second couplers is asymmetrical.

Example 22 is the system of Example 21 wherein the cross-section includes one or more concave portion and one or more convex portion.

Example 23 is the system of Example 16 wherein electrical connection is made between the one or more dilator electrodes and the control system when the one or more first couplers couples with the one or more second couplers.

Example 24 is the system of Example 16 wherein the one or more first couplers or the one or more second couplers includes a conductive surface.

Example 25 is the system of Example 16 wherein the one or more first couplers or the one or more second couplers includes an insulated housing configured to confine a brush.

Example 26 is the system of Example 16 wherein the one or more first couplers or the one or more second couplers includes a contact that is proud of an insulated surface.

Example 27 is the system of c Example 16 wherein the one or more first couplers or the one or more second couplers includes a plurality of insulated regions.

Example 28 is the system of Example 16 wherein the control system is configured to determine position of the dilator with respect to the sheath.

Example 29 is the system of Example 16 further comprising a display for displaying one or more anatomical images, parameters, and positioning information.

Example 30 is the system of Example 16 wherein the one or more sheath electrodes includes four sheath electrodes and the one or more dilator electrodes includes two electrodes.

Example 31 is a medical system having a sheath including an elongate body. The sheath elongate body includes a proximal portion and a distal portion. One or more sheath electrodes are located on the distal portion. A handle is attached to the proximal portion and includes one or more first couplers. The system includes a dilator having an elongate body. The dilator elongate body includes a proximal portion and a distal portion. One or more dilator electrodes are located on the distal portion. A hub is attached to the proximal portion of the dilator. One or more second couplers extend from the hub. The one or more first couplers is configured to receive the one or more second couplers. The system is configured to determine position of the dilator with respect to the sheath.

Example 32 is the system of Example 31 wherein the one or more first couplers or the one or more second couplers includes a conductive surface.

Example 33 is the system of Example 32 wherein the one or more first couplers or the one or more second couplers includes an insulated housing configured to confine a brush.

Example 34 is the system of Example 33 wherein the one or more first couplers or the one or more second couplers includes a contact that is proud of an insulated surface.

Example 35 is a medical system including a sheath having an elongate body. The elongate body includes a proximal portion and a distal portion. One or more sheath electrodes are located on the distal portion. A handle is attached to the proximal portion. The sheath includes one or more sockets. The system includes a dilator including an elongate body having a proximal portion and a distal portion. One or more dilator electrodes are located on the distal portion. A hub is attached to the proximal portion of the dilator. One or more plugs extend from the hub. The one or more sockets are configured to receive the one or more plugs. The system includes a display for displaying one or more anatomical images, parameters, and positioning information.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 illustrates a sheath in accordance with an embodiment of the present disclosure.

FIG. 3 illustrates a bottom view of the sheath in FIG. 2 in accordance with an embodiment of the present disclosure.

FIG. 4 illustrates a dilator in accordance with the present disclosure.

FIG. 5 illustrates the sheath of FIG. 2 and the dilator of FIG. 4 assembled in accordance with the present disclosure.

FIG. 6 illustrates electrical pathways in accordance with an embodiment of the present disclosure.

FIGS. 7A and 7B illustrate configurations for a sliding electrical connection in accordance with the present disclosure.

FIG. 8A illustrates a configuration for a sliding electrical connection in accordance with an embodiment of the present disclosure.

FIG. 8B illustrates impedance measured by the electrical connection of FIG. 8A.

FIGS. 9A-9C illustrate a configuration for a sliding electrical connection in accordance with an embodiment of the present disclosure.

FIG. 9D illustrates impedance measured by the electrical connection of FIGS. 9A-9C.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

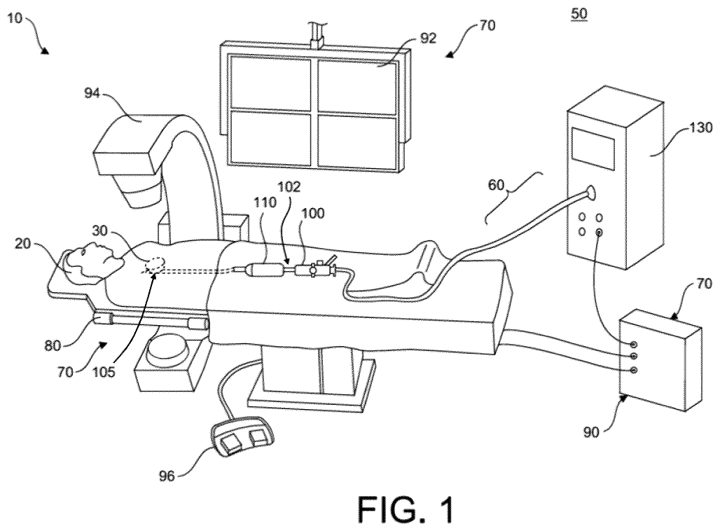

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an access system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The access system 60 includes a dilator 100 having a proximal portion 102 and a distal portion 105, an introducer sheath 110, and a console 130. In some aspects, the distal portion 105 includes a tapered region. Additionally, the access system 60 includes various connecting elements, e.g., cables, umbilicals, and the like, that operate to functionally connect the components of the access system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the dilator 100, in particular all or part of the distal portion 105 thereof, can be deployed to the specific target sites within the patient's heart 30. The dilator 100 is configured with a lumen such that a guiding device, for example a guidewire, or perforation device, for example an RF perforation device, can be inserted therein. In some aspects, a perforation device can be used to perform a transseptal crossing procedure within the heart 30.

The console 130 is configured to control functional aspects of the access system 60. In embodiments, the console 130 includes one or more controllers, microprocessors, and/or computers that execute code out of memory to control and/or perform the functional aspects of the access system 60. In embodiments, the memory can be part of the one or more controllers, microprocessors, and/or computers, and/or part of memory capacity accessible through a network, such as the world wide web. In embodiments, the console 130 can include pulse generator hardware, software and/or firmware configure to generate electrical pulses in predefined waveforms, which can be transmitted to electrodes positioned on a the dilator 100, guiding device, or perforation device to generate electric fields sufficient to achieve the desired clinical effect, for example ablation of target tissue through irreversible electroporation. In embodiments, the console 130 can deliver the pulsed waveforms in a monopolar or bipolar mode of operation, as will be described in further detail herein.

The EAM system 70 is operable to track the location of the various functional components of the access system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the dilator 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the dilator 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

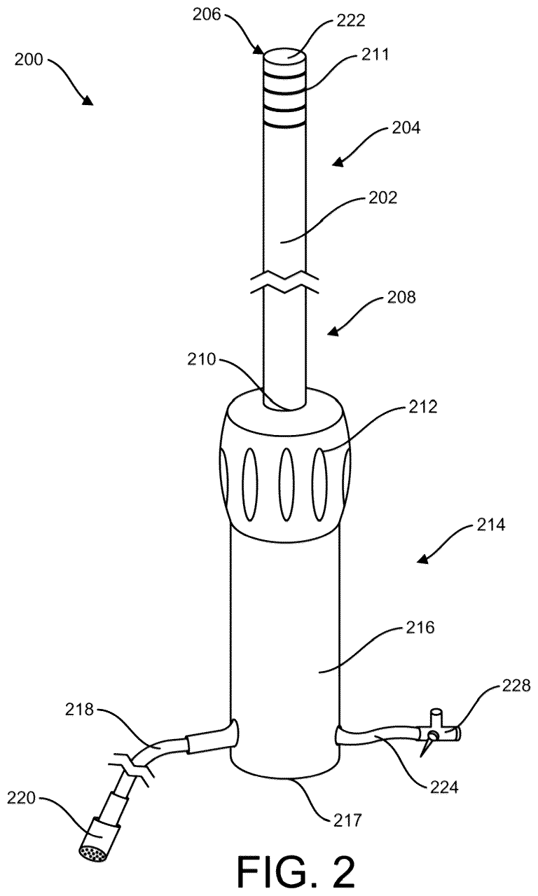

FIG. 2 illustrates a sheath 200 in accordance with an embodiment of the present disclosure. The sheath 200 includes an elongate hollow body 202 having a proximal portion 208 and a distal portion 204. The distal portion 204 includes one or more electrodes 211 positioned thereon and ends in a distal tip 206. In one aspect, the one or more electrodes 21 may include four electrodes. The one or more electrodes 211 can be evenly spaced or positioned at uneven intervals. The one or more electrodes 211 can be configured as surface electrodes capable of contacting tissue or fluid within a patient. The one or more electrodes 211 may be configured as part of an EAM system 70, to detect a parameter when in contact with the tissue or fluid, or to deliver energy to the tissue, such as RF energy in order to ablate the tissue.

The proximal portion 208 includes a proximal end 210 that removably couples to a handle 214. The handle 214 includes a stationary portion 216 configured to be held by a hand of a user and a rotatable knob 212 that is configured to adjust the shape of the distal portion 204. Rotatable knob 212 is connected to at least one control wire configured to deflect the distal portion 204 in a first direction when the knob 212 is rotated clockwise and to deflect the distal portion 204 in a second direction when the knob 212 is rotated counterclockwise. A lumen 222 extends from a handle proximal end 217 though the knob 212 and through the hollow body 202 to the distal tip 206. The lumen 222 is configured to receive a dilator or other elongate medical device.

The stationary portion 216 includes a cable 218 configured to couple with a system, for example an EAM system 70 or RF energy generator. The cable 218 includes a connector 220 that releasably engages a connector for the system. The stationary portion 216 also includes a conduit 224 which includes a fitting 226, such as a Luer connector. The conduit 224 is configured to allow for introduction of fluids into the lumen 222 or an additional lumen within the sheath 200. The fitting 226 is configured to removably connect to a syringe or other container for delivering a fluid through the conduit 224.

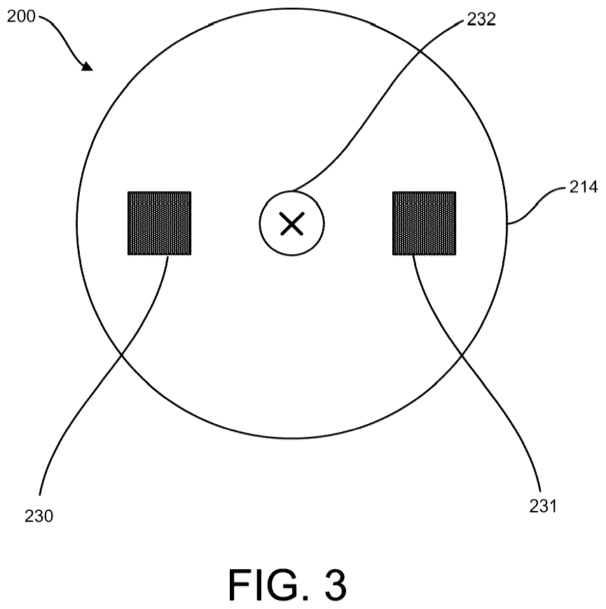

FIG. 3 illustrates a bottom view of the proximal end 217 of the sheath 200 in FIG. 2 in accordance with an embodiment of the present disclosure. The bottom surface of the proximal end 217 of the sheath 200 includes one or more first couplers 230 for mating with one or more second couplers associated with a dilator or other medical device. In some aspects the one or more first couplers 230 may include one or more recesses, sockets, openings, holes, apertures, raised portions, rods, shafts, plugs, surfaces, magnets, or other elements that may couple with a second coupler. When a corresponding second coupler is coupled with a first coupler 230, an electrical connection is formed.

As illustrated in FIG. 3, two couplers configured as sockets 230, 231 are located on opposite sides of a hemostatic valve 232 that closes the proximal end of lumen 222 extending through the sheath 200. The hemostatic valve 232 is configured to allow introduction of a fluid or a medical device, for example a dilator, into alignment within the sheath 200 while creating a fluid tight seal. The sockets 230, 231 are electrically isolated from the lumen such that there is no risk of signal interference or short circuiting. While two sockets 230, 231 are illustrated, it is understood that more or less sockets may be used in order to electrically connect more or less electrodes associated with a connected medical device to an external system, for example an energy generator or an EAM system 70.

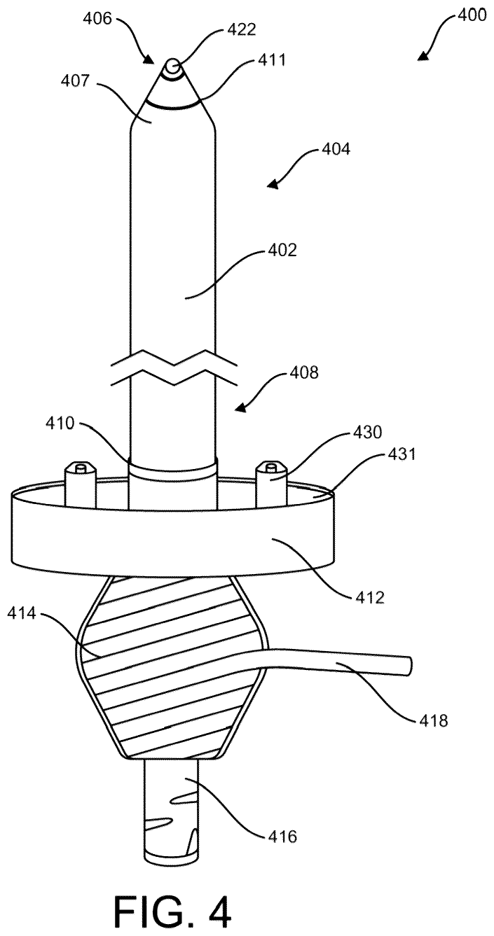

FIG. 4 illustrates a dilator 400 in accordance with an embodiment of the present disclosure. The dilator 400 is configured to be inserted into and couple with the sheath 200. The dilator 400 includes an elongate hollow body 402 having a proximal portion 408 and a distal portion 404. The distal portion 404 includes a tapered section 407 including one or more electrodes 411 positioned thereon. In the embodiment illustrated in FIG. 4, two electrodes 411 are included on the tapered section 407. In some embodiments, the dilator 400 may not include a tapered section.

The elongate hollow body 402 and ends in a distal tip 406. The one or more electrodes 411 can be evenly spaced or positioned at uneven intervals. The one or more electrodes 411 can be configured as surface electrodes capable of contacting tissue or fluid within a patient. The one or more electrodes 411 may be configured to detect a parameter when in contact with the tissue or fluid or to deliver energy to the tissue, such as RF energy in order to ablate the tissue.

The proximal portion 408 includes a proximal end 410 that removably couples to a hub 412. The hub 412 is attached to a handle 414. The handle 414 includes an extension 418 that aids in manipulation of the dilator 400 and acts as a reference for positioning the dilator 400. A connector 416 is positioned at the proximal portion of the handle 414. The connector 416 may include a Luer connector configured to removably connect to a syringe or other container. The connector 416 also allows for introduction of a medical device, such as a guidewire or RF perforation device, into a lumen 422 that extends from the connector 416 to the distal tip 406.

The hub 412 includes one or more second couplers 430. In some aspects, the one or more second couplers 430 take the form of plugs extending from an upper surface 431. In some aspects the one or more second couplers 230 may include one or more recesses, sockets, openings, holes, apertures, raised portions, rods, shafts, plugs, surfaces, magnets, or other elements that may couple with a first coupler.

The one or more second couplers 430 can include one or more plugs that are configured to mate with one or more sockets 230 on the sheath 200 when the dilator 400 is inserted into the sheath 200. When the one or more plugs 430 are inserted into the one or more sockets 230 an electrical connection is formed therebetween. This connection couples the one or more electrodes 411 to a system, for example an EAM system 70 or an energy generator, that is coupled to the sheath 200 and electrodes 211. Additionally, when the one or more plugs 430 are located in the one or more sockets 230, relative rotation between the sheath 200 and dilator 400 is resisted or prevented. That is, a rotational lock is formed between the sheath 200 and the dilator 400. As such, the sheath 200 and the dilator 400 can be rotated together by virtue of rotating either the sheath 200 or the dilator 400.

In some aspects, a snap or locking feature may be associated with the hub 412 or the bottom surface of the proximal end 217 of the sheath 200. The snap or locking feature may ensure mechanical coupling between the sheath 200 and the dilator 400. In some aspects, a snap or locking feature may be associated with the one or more plugs 430 or the one or more sockets 230.

The one or more plugs 430 include a shape that corresponds to the one or more sockets 230. The one or more plugs 430 are elongate and have a cross-section substantially similar to the opening of the one or more sockets 230. In some aspects the one or more plugs 430 may have a rectangular or square cross-section. In other aspects, the cross-section may be configured such that only one insertion orientation is possible. For example, the one or more sockets 230 or the one or more plugs 430 may include an asymmetric cross-section such that only one or more plugs 430 can be received in the one or more sockets 230 in only one orientation. In some aspects, the asymmetric cross-section may include one or more concave portion or one or more convex portion.

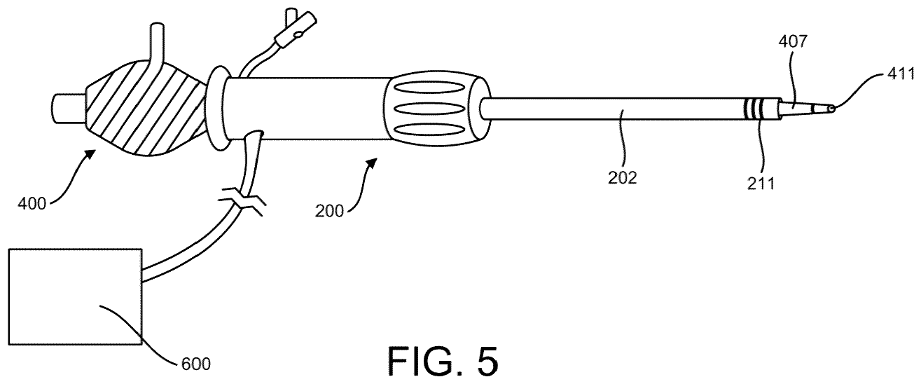

FIG. 5 illustrates the sheath 200 of FIG. 2 and the dilator 400 of FIG. 5 assembled in accordance with the present disclosure. In FIG. 5 the dilator 400 is coupled to the sheath 200. The tapered section 407 of the dilator 400 extends from the distal end of the sheath 200. In this configuration the one or more electrodes 411 are exposed. Additionally, the one or more electrode 211 of the sheath are exposed. When coupled together, each of the electrodes are electronically coupled to the control system 600. Control system 600 can be an EAM system 70 or an energy generator for ablation.

In some aspects, the control system 600 includes a display. The display is configured to display anatomical images, such as illustrations of a heart, measured parameters, and/or positioning or orientation information associate with the sheath 200 and dilator 400. Positioning or orientation information may include information related to relative distances between components, end positioning, curvature or shape, or other positioning or orientation information. The positioning or orientation information may include an image or illustration of the sheath 200 and/or the dilator 400. In some aspects, the control system 600 is configured to recognize the sheath 200 and dilator 400 as one device with known distances between electrodes. This allows for a representative image of the position or orientation of the joined sheath 200 and dilator 400 to be displayed. In this configuration, the display may display information related to all electrodes, for example six total electrodes. In some aspects, the control system 600 is configured to recognize when the sheath 200 and dilator 400 are no longer coupled when the dilator 400 is removed from the sheath 200. In this configuration, the display may display information related only to electrodes associated with the sheath 200, for example four total electrodes.

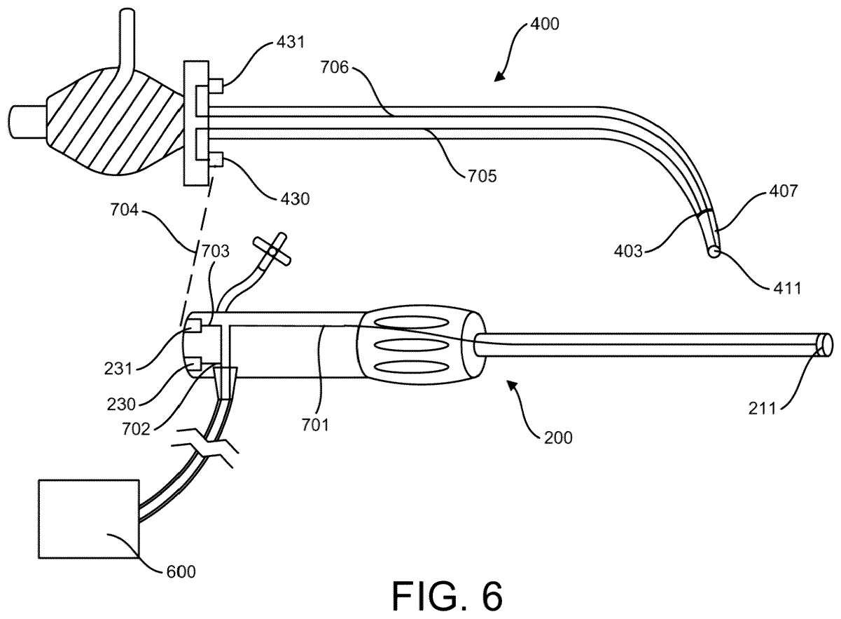

FIG. 6 illustrates an electrical pathway in accordance with an embodiment of the present disclosure. Electrical pathways may include wires or other conductors such as thin film traces. In FIG. 6, the sheath 200 and dilator 400 are uncoupled. In the embodiment of FIG. 6, the sheath 200 is illustrated as having a single distal electrode 211 and the dilator 200 is shown having a distal electrode 411 and a proximal electrode 413.

The sheath 200 is connected to the control system 600 which can be an EAM system 70 or an energy generator. A first pathway 701 connects the distal electrode 211 to the system 600. A second pathway 702 connects a first socket 230 to the system 600, and third pathway 703 connects a second socket 231 to the system 600. Upon insertion of the dilator 400 into the sheath 200, an electrical coupling (indicated as 704) is formed between the first socket 230 and a first plug 430 of the dilator 400 and between the second socket 231 and a second plug 431 of the dilator 400. A fourth pathway 705 connects the proximal electrode 413 to the second plug 431, and a fifth pathway 706 connects the distal electrode 411 to the first plug 430. Therefore, electrical coupling between the distal electrode 411 and the control system 600 and the proximal electrode 413 and the control system 600 is achieved by insertion of the dilator 400 into sheath 200 such that the first plug 430 and the second plug 431 mate with the first socket 430 and the second socket 231 respectively.

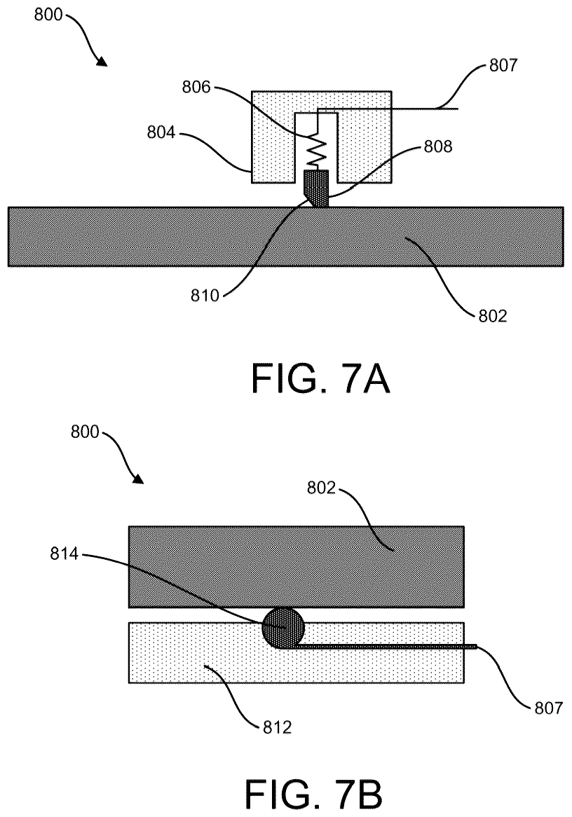

FIGS. 7A and 7B illustrate configurations for a sliding electrical connection 800 between concentric members, for example a plug of a dilator and a socket of a sheath, in accordance with the present disclosure. FIG. 7A illustrates a sliding electrical connection 800 having a constrained spring 806 and brush 808. An insulated housing 804 constrains the spring 806 and brush 808 to limit the direction of movement of the spring 806 and brush 808 upon insertion of a plug into a socket. The housing 804 limits horizontal movement of the spring 806 and bush 808 and allows the spring 806 and brush 804 to move freely in a vertical direction. The brush 808 can include a beveled edge 810 to facilitate sliding along a conductive surface 802. In one aspect, the conductive surface 802 may form a portion of a plug. In another aspect, the conductive surface 802 may form a portion of a socket. A conductor 807 extends from the brush 808 for electrical connection between components.

FIG. 7B illustrates a sliding electrical connection 800 having a fixed raised contact 814. The contact 814 can be a conductive material, such as a metal, and is fixed in an insulated surface 812. In some aspects, a spring may be attached to the contact 814. The contact 814 makes an electrical connection with a conductive surface 802 when translating past the conductive surface 802. In one aspect, the conductive surface 802 may form a portion of a plug. In another aspect, the conductive surface 802 may form a portion of a socket. A conductor 807 extends from the contact 814 for electrical connection between components.

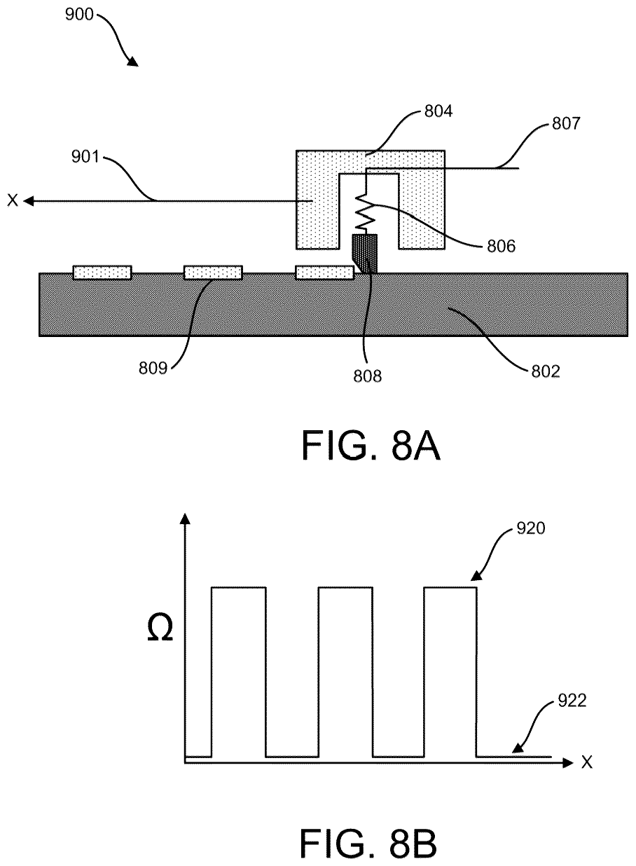

FIG. 8A illustrates a configuration for a sliding electrical connection 900 in accordance with an embodiment of the present disclosure configured to detect position of a sheath in relation to a dilator. In FIG. 8A, there is shown an insulated housing 804 that constrains a spring 806 and brush 808 similar to FIG. 7A. However, in FIG. 8A the conductive surface 802 includes a plurality of insulated regions 809 or regions having an electrical impedance different than that of conductive surface 802.

FIG. 8B illustrates impedance measured by the brush 808 as it passes over the conductive surface 802 and insulated regions 809. As illustrated in FIG. 8B, when the brush 808 is in contact with the conductive surface 802 a low impedance is measured at 922. When the brush 808 passes over an insulated region 809 a high impedance is measured at 920. As such, as the bush 808 moves along the conductive surface 802 in the direction of arrow 901, a series of high 920 and low 922 impedances are measured. By using the measured impedances, and the known spaces between the insulated regions 809, it is possible to determine the location of a dilator in relationship to a sheath. This allows for an indication of when the tapered end of the dilator is exposed completely, partially, or not at all from an end of the sheath.

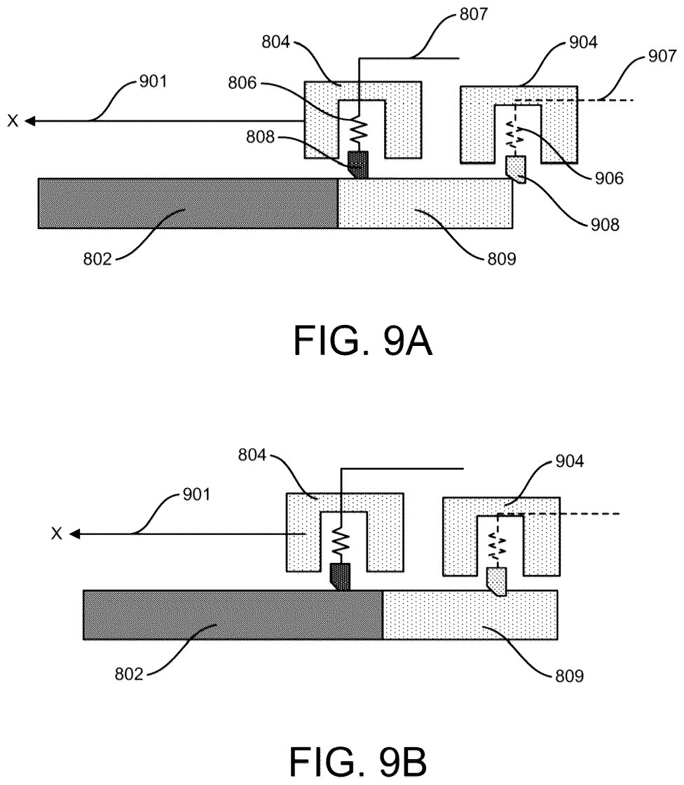

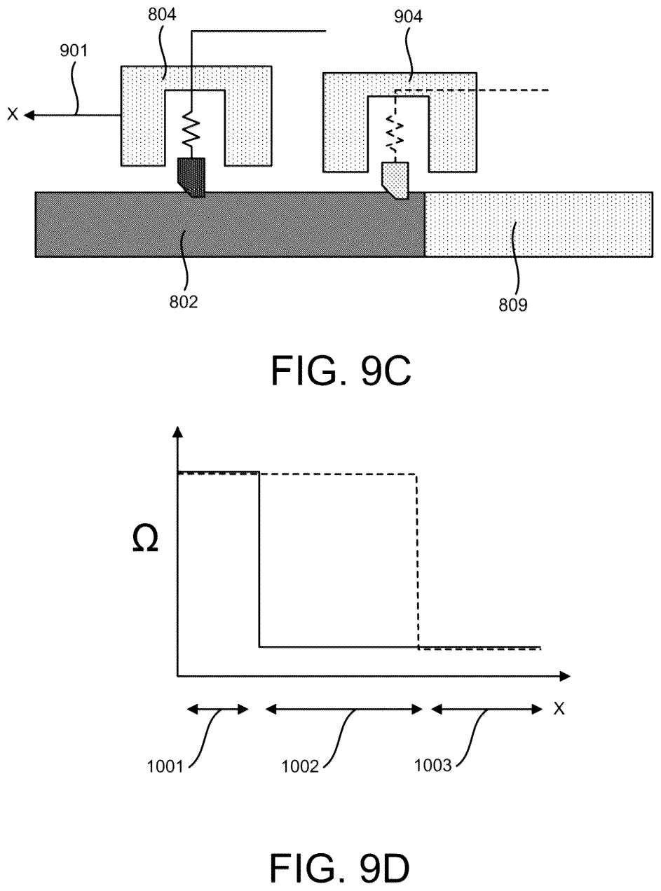

FIGS. 9A-9C illustrate another configuration for a sliding electrical connection in accordance with an embodiment of the present disclosure that allows for detecting positioning of a dilator in relationship to a sheath. In FIGS. 9A-9C, there is shown a first insulated housing 804 that constrains a first spring 806 and a first brush 808 and a second insulated housing 904 that constrains a second spring 806 and a second brush 908. In a first aspect, the first insulated housing 804 and the second insulated housing 904 are associated with a plug. In a second aspect, the first insulated housing 804 and the second insulated housing 904 are associated with a socket.

The first brush 808 and the second brush 908 are configured to move relative to a conductive surface 802 and an insulated region 809. As the first brush 808 and the second brush 908 translate along the surface, changes in impedance can be monitored as indicated in FIG. 9D. In one aspect, the first insulated housing 804 and the second insulated housing 904 remain stationary as the conductive surface 802 and the insulated region 809 are moved. In another aspect, the conductive surface 802 and the insulated region 809 are remain stationary as the first insulated housing 804 and the second insulated housing 904 translate.

In FIG. 9A both the first brush 808 and the second brush 908 are positioned over an insulated region 809. As such, FIG. 9D illustrates both brushes measuring a high impedance at 1001. In FIG. 9B the first brush 808 is positioned over the conductive surface 802 and the second brush 908 is positioned over an insulated region 809. As such, FIG. 9D illustrates measuring a high impedance as well as a low impedance at 1002. In FIG. 9C both the first brush 808 and the second brush 908 are positioned over the conductive surface 802. As such, FIG. 9D illustrates both brushes measuring a low impedance at 1003. By using the measured impedances, and the lengths of conductive surfaces 802 and insulated regions 809, it is possible to determine the location of a dilator in relationship to a sheath.

In some aspects, the sheath 200 and dilator 400 can be packaged as a kit and be ready for use right out of the package. The kit may also include a RF perforation or puncturing device 110. Alternatively, the kit may include a plurality of sheaths or dilators each having different pre-shaped portions for use in various procedures.

In some aspects, the sheath 200 or dilator 400 may include one or more markers along a portion thereof for identifying a position or location during use using an imaging modality.

In some aspects, the sheath 200 or dilator 400 may include a plurality of cuts machined into the wall, for example by laser cutting. The shape and positioning of the cuts can allow for a transition in flexibility from a proximal portion to distal portion. The cuts may include a broken spiral configuration or may be positioned substantially orthogonal to a longitudinal axis of the sheath 200 or dilator 400. In some aspects, there may be a single cut that winds around an axis with a wider spacing between loops at the proximal portion and a larger spacing at the distal portion. The spacing and size of the cuts can be varied to achieve different flexibilities along the length of the sheath 200 or dilator 400.

In some aspects, the sheath 200 or dilator 400 may be formed of a shape memory material, such as a shape memory polymer or a shape memory metal. This would allow the sheath 200 or dilator 400 to have a first shape at a first temperature, and a second shape at a second temperature. Shape transition may be initiated by inserting a heated solution into the sheath 200 or dilator 400 or using electricity to heat a portion of the sheath 200 or dilator 400.

In some aspects, rather than a dilator, the sheath 200 may be electrically and mechanically coupled with a catheter, sheath, lead, needle, or other elongated medical device that includes a hub with one or more second couplers as described above.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical system, comprising:
a sheath including an elongate body having a proximal portion and a distal portion;
one or more sheath electrodes located on the distal portion;
a handle attached to the proximal portion; and
one or more first couplers capable of electrically coupling to a control system;
a dilator including an elongate body having a proximal portion and a tapered distal portion;
one or more dilator electrodes located on the tapered distal portion;
a hub attached to the proximal portion of the dilator; and
one or more second couplers located on the hub, wherein the one or more first couplers is configured to electrically couple with the one or more second couplers.

2. The system of claim 1, wherein the sheath elongate body includes a lumen extending from the proximal portion to the distal portion and is configured to receive the dilator.

3. The system of claim 1, wherein the hub includes an upper surface, and the one or more second couplers extends from the upper surface, and wherein a rotational lock is formed between the sheath and the dilator when the one or more first couplers are coupled with the one or more second couplers.

4. The system of claim 1, wherein the one or more first couplers and the one or more second couplers have corresponding shapes.

5. The system of claim 1, wherein a cross-section of the one or more first couplers and the one or more second couplers is symmetrical.

6. The system of claim 1, wherein a cross-section of the one or more first couplers and the one or more second couplers is asymmetrical.

7. The system of claim 6, wherein the cross-section includes one or more concave portion and one or more convex portion.

8. The system of claim 1, wherein electrical connection is made between the one or more dilator electrodes and the control system when the one or more first couplers couples with the one or more second couplers.

9. The system of claim 1, wherein the one or more first couplers or the one or more second couplers includes a conductive surface.

10. The system of claim 1, wherein the one or more first couplers or the one or more second couplers includes an insulated housing configured to confine a brush.

11. The system of claim 1, wherein the one or more first couplers or the one or more second couplers includes a contact that is proud of an insulated surface.

12. The system of claim 1, wherein the one or more first couplers or the one or more second couplers includes a plurality of insulated regions.

13. The system of claim 1, wherein the control system is configured to determine position of the dilator with respect to the sheath.

14. The system of claim 1, further comprising a display for displaying one or more anatomical images, parameters, and positioning information.

15. The system of claim 1, wherein the one or more sheath electrodes includes four sheath electrodes and the one or more dilator electrodes includes two electrodes.

16. A medical system, comprising:
a sheath including an elongate body having a proximal portion and a distal portion;
one or more sheath electrodes located on the distal portion;
a handle attached to the proximal portion; and
one or more first couplers;
a dilator including an elongate body having a proximal portion and a distal portion;
one or more dilator electrodes located on the distal portion;
a hub attached to the proximal portion of the dilator; and
one or more second couplers extending from the hub, wherein the one or more first couplers is configured to receive the one or more second couplers; and
wherein the system is configured to determine position of the dilator with respect to the sheath.

17. The system of claim 16, wherein the one or more first couplers or the one or more second couplers includes a conductive surface.

18. The system of claim 17, wherein the one or more first couplers or the one or more second couplers includes an insulated housing configured to confine a brush.

19. The system of claim 18, wherein the one or more first couplers or the one or more second couplers includes a contact that is proud of an insulated surface.

20. A medical system, comprising:
a sheath including an elongate body having a proximal portion and a distal portion;
one or more sheath electrodes located on the distal portion;
a handle attached to the proximal portion; and
one or more sockets;
a dilator including an elongate body having a proximal portion and a distal portion;
one or more dilator electrodes located on the distal portion;
a hub attached to the proximal portion of the dilator; and
one or more plugs extending from the hub, wherein the one or more sockets is configured to receive the one or more plugs; and
a display for displaying one or more anatomical images, parameters, and positioning information.
